(19) United States
(12) Patent Application Publication
(10) Pub. No.: US 2018/0122518 A1
(43) Pub. Date: May 3, 2018

(54) METHOD FOR MONITORING AND CONTROLLING PATIENT PARAMETERS AND TRANSMITTING MEDICAL INFORMATION AND A SYSTEM FOR CARRYING OUT THE METHOD

(71) Applicant: MEDINICE SPOLKA AKCYJNA, Kielce (PL)

(72) Inventors: Sanjeev CHOUDHARY, Warsaw (PL); Sebastian STEC, Warszawa (PL)

(21) Appl. No.: 15/566,677

(22) PCT Filed: Sep. 24, 2015
(86) PCT No.: PCT/PL2015/000147

§ 371 Date: Oct. 13, 2017

(30) Foreign Application Priority Data
Apr. 14, 2015 (PL) P.412000

(51) Int. Cl. G16H 80/00; H04N 7/15

(52) U.S. Cl. CPC G16H 80/00; H04N 7/15

(57) ABSTRACT

The method of monitoring and interactive control of patient parameters and communicating the medical information includes video image recording from at least one medical device monitor with a camera, video streaming, application streaming, displaying an image recorded on a computer screen or via a dedicated application on a mobile device, archiving of the video image, and providing a two-way communication. The subject is also a system for implementing the method.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention is a method for monitoring and interactive controlling of patient parameters and transmitting medical information and a system for carrying out the method, which focuses on several aspects, such as telemonitoring, improving the flow of medical information and remote control of patient parameters.

### 2. Description of Related Art Including Information Disclosed Under 37 CFR 1.97 and 37 CFR 1.98

Modern medical treatments base on the use of software running on multiple computers and additional electronic supporting systems (i.e. communication, observation, diagnostics). The main purpose of using computer techniques is to support tile medical procedure, raise its quality and safety, and the ability to record and analyse the parameters. Together with the development of the computerization of the operation of treatments, surgeries, medical procedures, a problem occurred with a lack of integration of recorded and monitored parameters, making their observation and quick interpretation by medical personnel a more and more difficult and onerous task.

At the same time the development of technologies designed to support physicians creates a need for additional assistance of specialists and experts, and these are limited human resources. Repeatedly, diagnostics and performance of procedures are associated with the occurrence of rare diseases, as well as diagnostic and therapeutic difficulties due to the limitations of knowledge and skills of local personnel. In such cases, it becomes necessary to carry out intra-procedural consultation, the lack or delay of which can cause additional risk for the patient (e.g. whether it is possible to use an additional X-ray radiation dose, wrong diagnosis and undertaken treatment or therapeutic technique), procedure and treatment delay (seeking ways of problem solving) or its failure (ending the procedure at diagnostic stage, postponement of therapeutic procedure), or increase in the cost of treatment.

The literature describes a number of solutions describing attempts to solve problems related to communication between medical devices, improvements of medical treatments through the use of devices communicating with each other and with medical devices.

Solution known from the prior art is described in the document US2003109904A, and it concerns the system of transmission of medical information, including: medical device with installed software; centre of communication software connected to the medical device to interact with the medical device, wherein the centre of the software communicates with the medical device, regardless of the software version installed in the medical device.

The US2009112630A application also describes a system communicating and collecting data from many different medical devices, wherein each of the medical devices includes an output port. The system includes a local data collection module comprising a first controller, a receiver connected to the first controller and operating to receive local wireless short-range signal, a transceiver device connected to the first controller and operating to communicate wirelessly with access to an Ethernet network of health care facility, and multiple adapters of medical devices, each of which includes a second controller, which works in the same way as the first one, wherein the local communication module communicates wirelessly with at least one receiver of data received wirelessly from each medical device adapter to the system by the access point in order to automatically enter the patient medical entry connected to a plurality of medical devices.

In published documents no communication system was found that would aim to improve the efficiency and safety of the performance of treatments and medical procedures by improving the interpersonal communication and optimal use of diagnostic and therapeutic techniques for the efficient conducting of intra-procedural consultation or remote control of procedures performance, use of selected techniques or supervision of the whole operation by an expert.

### BRIEF SUMMARY OF THE INVENTION

The system of the invention allows:

Integration in one place of the registered and monitored parameters during performed treatments, medical procedures,
Sharing of the integrated above information by means of video transmission;
Allowing remote (distance) interpersonal and inter-procedural consultations; and
Allowing the exchange of experiences.

The essence of the invention is a method of monitoring and interactive controlling of patient parameters and communicating the medical information comprising the following steps of:
a) video image recording from at least one medical device monitor with a camera
b) video streaming
c) application streaming
d) displaying an image recorded in step a) on a computer screen or via a dedicated application in a mobile device
e) archiving the video image recorded in step a)
f) providing a two-way communication

Preferably, as a camera an analogue or digital camera is used.

Preferably, the video image transfer is effected by means of the Internet, LAN network, WLAN.

Preferably, chat, voice connection, video connection is used as a form of providing two-way communication.

A second object of the invention is a system for monitoring patient parameters and transmitting medical information comprising:

a) at least one camera incorporated into the IT network

b) video streaming server

c) a computer with an access to the IT network

d) application server

e) mobile device with installed mobile application

Preferably, the system additionally comprises a router.

Preferably, the camera records the image from at least one medical device monitor.

Preferably, the image recorded by the camera is transmitted to the authorized recipients, wherein the computer in the local network, via a router, gains an access to the web application served from an application server, in which video streaming is presented, or directly downloads the video streaming shown in the client application installed on the mobile device.

The beneficial effects of the invention are:
Raising and improving the ergonomics of performed treatments, medical procedures;
Integration of multiple medical parameters fed from multiple sources in a single location so that the monitoring and interpretation of many medical parameters within the performed treatment, medical procedure are easier to perform;
The ability to provide monitoring of medical parameters and a view from ongoing treatments, medical procedures at a distance;
Allowing recording, storage, processing and management of recorded treatments, medical procedures, which would create a medical database with an easy access and search capabilities according to specific parameters; and
Ease and versatility of implementation and access to the system according to the invention as well as results, records of its work.

Thanks to the above assumptions the system according to the invention is not only a solution revolutionizing the handling of the treatments and medical procedures but also, thanks to an optimal architecture, easy accessibility and optimized costs of solution implementation, it becomes a commonly and widely available solution for medical facilities and hospitals.

The system according to the invention is available through the web browser and dedicated mobile applications available for mobile devices with operating systems i.e.: iOS since version 6.x, Android since version 2.3.

Access to the system according to the invention is password-protected. As a result, on the one hand access to the console will be common, while on the other hand restricted only to authorized persons, who will be able to have access to the medical content. The system according to the invention is a medical application creating new opportunities to transmit knowledge, between the operating room and an expert located in a different location.

### BRIEF DESCRIPTION OF THE SEVERAL VIEWS OF THE DRAWINGS

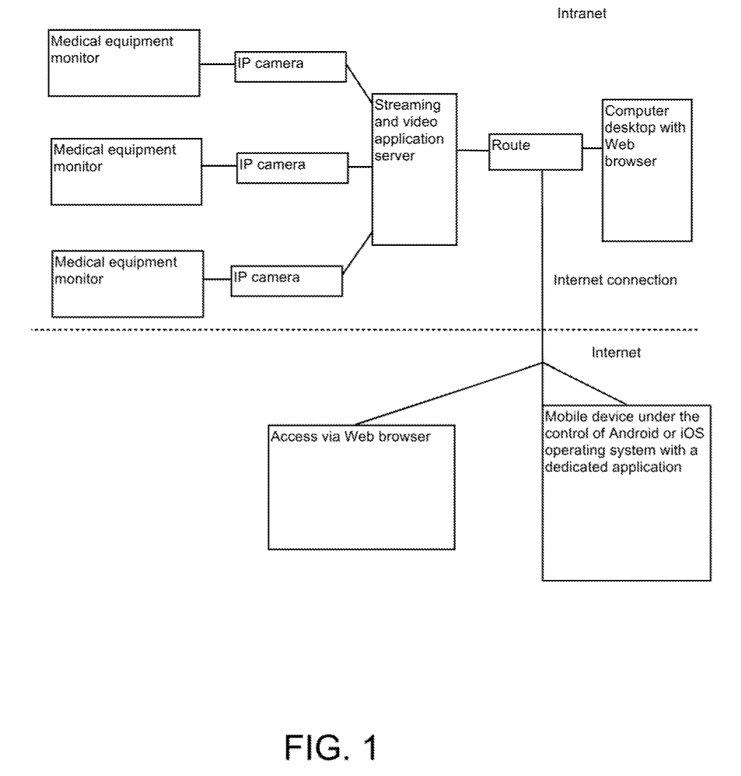

The invention has been closely described in the following embodiment and on the drawing, in which FIG. 1 shows schematic view of an architectural diagram of a system for monitoring patient parameters and transmitting medical information.

### DETAILED DESCRIPTION OF THE INVENTION

The system according to the invention consists of three basic elements:

1. Devices for Recording Image from Medical Devices

Devices that are designed to register multiple medical parameters recorded during the performance of treatments, medical procedures are high-resolution IP cameras, which record the views from the individual medical monitors of peripheral devices —computers. The cameras are installed above monitors in a room separated from the operating room, monitoring the course of the treatment, medical procedure. In a preferred embodiment, one camera is installed for each monitor giving a preview of medical parameters, recording the particular medical parameter. By recording through independent, professional, high-resolution camera with fixed IP, the monitoring of medical parameters of the system according to the invention provides stability, quality and reliability at the highest possible level. Cameras, thanks to the constant IP connection in a secure, wired way transmit the view from a preview of a particular monitor-medical parameter to the application server. The application server is a central unit of the system, responsible for the collection, processing and sharing of medical data.

2. Processing and Information Management System

The processing and information management system consists of the application server with the devices to transmit data. The application server manages both video streaming and other system data. Data transmission devices are router with integrated firewall, permanent Internet link and Ethernet network cabling. On the application server side there is also a system database, as well as the WWW applications and dedicated mobile applications backend.

Video streaming is a delivery of multimedia information through the network (internet or intranet). The multimedia data transmitted by this method are compressed using one of the compression formats. The most popular compression formats include MPEG-4, H.264, QuickTime. However, despite the video material compression, it still requires a relatively high Internet network bandwidth or data communication network. It is estimated that for the streaming of Full HD video at 30 fps a link with a capacity of 6 Mbit is required. Such bandwidth is sufficient for a single recipient of so transferred video. If the network bandwidth is not sufficient for video streaming, techniques are used which allow depending on requirements: to limit the number of frames per second (fps) without compromising image quality, to reduce image quality (lower the resolution) but without limiting the fps, to limit the quality and fps to keep the continuity of video playback. In case of video transmission in real time, video buffering technique is also used to maintain the continuity of video playback at the expense of a slight delay in transmission streaming. TCP/IP and http protocols are most commonly used to transmit video materials.

In the process of video streaming the following elements take part:
video camera as an image and sound recording device;
video streaming server—a computer with appropriate parameters and appropriate software for video streaming installed;
Internet connection with appropriately high bandwidth; and
a client device of the final recipient of transmitted video material—e.g. Web browser, application for tablet or phone.

The video camera records the image in a resolution and number of fps fixed for it. The video streaming server retrieves the recorded image from the camera and delivers it to the final recipient. The streaming server, depending on the configuration, uses various techniques described above to provide video material according to the expectations of the recipient.

3. Of Client Application.

Access to the system according to the invention is possible via:

an application that runs in a Web browser;

a dedicated mobile application installed on a mobile device; and

a video preview of the system view on the monitor screen.

Access to the system according to the invention is through a web browser.

Access to the system will be possible from the local level of a PC-type computer via a web browser, i.e. e.g.: Firefox since version 20.x, Google Chrome since version 28.x, Internet Explorer since version 8.x.

Thereby, access to the system according to the invention from the final user level, regardless of the level and nature of his permissions, will be quick and easy, will not require installing locally in the environment of a computer (workstation) of additional software etc.

The system according to the invention is also available as a dedicated application for mobile devices running under operating systems, i.e. e.g.: iOS since version 6.x or Android since version 2.3.

Explanation of Basic Terms:

Camera is e.g. an IP camera recording video image and transmitting it via TCP/IP protocol.

IP camera records image at a resolution of 1920×1080, compresses (e.g. in MPEG-4 format) and shares via the RJ-45 link in the TCP/IP protocol. Streaming server connected via Ethernet cable to the IP camera downloads the picture from the camera and then transfers it to the authorized final recipients. Through the router, the computer desktop in the local network accesses web applications served from the application server, in which streaming video is presented. Recipients on the Internet (mobile devices, Web browsers) through the Internet connection connected to the router, access the web applications, where the streaming is presented, or, if an application is dedicated to the mobile device (tablet, smartphone), they directly download the video streaming shown in a client application installed on a mobile device.

Video and application streaming server is a physical machine with software needed for video streaming from IP cameras and Web application server for viewing the video streaming.

His tasks include video streaming from the cameras, storage of recorded video materials, hosting the backend of Web applications and a dedicated application for mobile devices.

Router is an Ethernet router communicating the devices displaying the video streaming or operating on Web application or dedicated one with the video streaming server.

Video streaming from streaming server to applications installed on mobile devices or web applications on client computers is shared via router. The router also shares the connection to the application server. In both cases, communication takes place in both the local network and the Internet.

Computer is a physical machine working in the local subnet presenting video streaming.

In accordance with the system requirements, at least one computer workstation is available in the local network. In this workstation the user using a Web application will have access to the video streaming from the camera or cameras.

Web browser, mobile device are terminal equipment in the Internet network, where, via appropriate web application or dedicated one (for mobile devices such as tablet, smartphone), video streaming is presented.

Medical equipment monitor is an LCD monitor connected to the medical equipment, whose image is recorded using IP cameras.

Permanent Internet Access

The system allows users from non-local network, connected to the Internet anywhere in the world to use the system. Therefore, the system services (video streaming, web application) must be available on the Internet through a permanent connection.

Internet connection requirements:
symmetrical with a capacity of min 25 Mbit (assuming that we transmit the image from max. 3 Full HD cameras); and
at least one public IP address.

Software Infrastructure (Software)

Application server can be e.g. Apache 2 web server in conjunction with PHP 5.5. MySQL is used as a database server. SSL 256 bit certificate was used to secure the communication of client applications to the server. The backend of Web application and application dedicated for mobile devices is on the application server side. MySql database server is designed to store user data and content presented in Web applications and dedicated one for mobile devices.

Video Streaming Server Software

The software for transferring images from video cameras to client applications (web and dedicated application for mobile devices).

1. A method of monitoring and interactive control of patient parameters and transmitting medical information, the method comprising the following steps:
g) video image recording from at least one medical device monitor with a camera;
h) video streaming;
i) application streaming;
j) displaying an image recorded in step a) on a computer screen or via a dedicated application in a mobile device;
k) archiving of video image recorded in step a); and
l) providing a two-way communication.

2. The method according to claim 1, wherein an analogue or digital camera is used as a camera.

3. The method according to claim 1, wherein the video image transfer is by Internet, LAN network, or WLAN.

4. The method according to claim 1, wherein chat, voice connection, video connection is used as a form of providing two-way communication.

5. A system for monitoring patient parameters and transmitting medical information, the system comprising:
at least one camera incorporated into the IT network;
video streaming server;
a computer with an access to the IT network;
application server; and
mobile device with installed mobile application.

6. The system according to claim 5, further comprising: a router.

7. The system according to claim 5, wherein the camera records the image from at least one medical device monitor.

8. The system according to claim 5, wherein the image recorded by the camera is transmitted to the authorized recipients, wherein the computer in the local network, via a router, gains an access to the web application served from an application server, in which video streaming is presented, or directly download the video streaming shown in the client application installed on the mobile device.
